(19) United States
(12) Patent Application Publication
(10) Pub. No.: US 2008/0167669 A1
(43) Pub. Date: Jul. 10, 2008

(54) Vessel tensioning handle and method of vessel harvesting

(76) Inventors: Cynthia T. Clague, Minnetonka, MN (US); Michael J. Hobday, Lino Lakes, MN (US); Raymond W. Usher, Coon Rapids, MN (US); Roderick E. Briscoe, Rogers, MN (US); Katherine S. Olig, Eden Prairie, MN (US); Ana R. Menk, Minneapolis, MN (US); Christopher P. Olig, Eden Prairie, MN (US); Eric A. Meyer, Andover, MN (US); Steven C. Christian, New Brighton, MN (US); Thomas P. Daigle, Corcoran, MN (US); Robert H. Reetz, Rockford, MN (US); Jeffrey D. Sandstrom, Forest Lake, MN (US); James R. Keogh, Maplewood, MN (US); Matthew D. Bonner, Plymouth, MN (US); Scott E. Jahns, Hudson, WI (US); Philip J. Haarstad, Chanhassen, MN (US)

Correspondence Address: MEDTRONIC, INC., 710 MEDTRONIC PARKWAY NE, MINNEAPOLIS, MN, 55432-9924, US

(21) Appl. No.: 11/974,906

(22) Filed: Oct. 16, 2007

Related U.S. Application Data
(60) Provisional application No. 60/852,020, filed on Oct. 16, 2006.

(51) Int. Cl. A61B 17/32; A61B 17/22

(52) U.S. Cl. 606/159

(57) ABSTRACT

Embodiments of the invention provide a vessel tensioning handle and method of placing tension on a section of a vessel being harvested. The handle can include a housing, a bobbin assembly, a vessel tensioning tape, and a tensioning device member. The tensioning device member can be coupled to a cannula that is coupled to the vessel. The method can include using the vessel tensioning tape to place tension on the section of the vessel as the cutting device is advanced over the vessel.

### RELATED APPLICATIONS

This application claims priority under 35 U.S.C. §119 to U.S. Provisional patent application Ser. No. 60/852,020, filed on Oct. 16, 2006, the entire contents of which is hereby incorporated by reference.

### FIELD OF THE INVENTION

This invention relates generally to biomedical systems and methods. More specifically, the invention relates to systems and methods for harvesting a vessel section.

### BACKGROUND

Heart disease, specifically coronary artery disease, is a major cause of death, disability, and healthcare expense in the United States and other industrialized countries. A common form of heart disease is atherosclerosis, in which the vessels leading to the heart are damaged or obstructed by plaques containing cholesterol, lipoid material, lipophages, and other materials. When severely damaged or obstructed, one or more of the vessels can be bypassed during a coronary artery bypass graft (CABG) procedure. CABG surgery is performed about 350,000 times annually in the United States, making it one of the most commonly performed major operations.

To prevent rejection, the graft material is preferably a blood vessel harvested from elsewhere within a patient's body. The most frequently used bypass vessel is the saphenous vein from the leg. Because the venous system of the leg is redundant, other veins that remain within the patient's leg are able to provide return blood flow following removal of the saphenous vein.

Various methods have been used to harvest the saphenous vein. Until recently, the typical procedure involved making a single long incision that overlies the entire length of the vein, extending from a patient's groin to at least the knee and often to the ankle. This method results in substantial postoperative pain, with patients frequently complaining more of discomfort at the site of the leg vein harvesting than of pain from their CABG surgery wound. In addition, such an extensive incision site is subject to infection and delayed healing, especially in patients with poor circulation, which not infrequently accompanies coronary artery disease. The disfiguring scar from such a large incision is also of concern to some patients.

Less invasive procedures are preferred, and surgical devices and techniques now exist that allow the saphenous vein to be harvested through one or more small, transverse incisions along the length of the vein, generally using an endoscope. Endoscopic procedures yield reduced wound complications and superior cosmetic results compared with traditional methods of vein harvesting. However, this procedure requires considerable manipulation of the vein, has a high conversion rate when visualization is obscured by bleeding or the procedure is taking too long and often requires stitches to repair the vein following harvest. Further, it is generally tedious, time consuming, and relatively complex, requiring extensive accessory equipment and a substantial learning curve for the surgeon.

### SUMMARY

Some embodiments of the invention provide a vessel tensioning handle for use with a cutting device and a cannula for harvesting a section of a vessel. The handle can include a housing adapted to be coupled to the cutting device and a bobbin assembly positioned within the housing. The handle can also include a vessel tensioning tape wrapped around the bobbin assembly. The handle can further include a tensioning device member coupled to the vessel tensioning tape. The tensioning device member can be adapted to be coupled to the cannula. The cannula can be coupled to the vessel. The vessel tensioning tape can place tension on the section of the vessel as the cutting device is advanced over the vessel.

Embodiments of a vessel harvesting method of the invention include making a first incision at a proximal end of the section of the vessel, and making a second incision at a distal end of the section of the vessel. The method can also include inserting a cannula into the proximal end of the vessel, and securing the proximal end of the vessel to the cannula. The method further includes coupling a vessel tensioning tape to the cannula, applying tension to the vessel, and advancing a cutting device over the vessel.

### DESCRIPTION OF THE DRAWINGS

FIGS. 1A-1F are illustrations of vessel tensioning handles for use in harvesting a vessel section according to some embodiments of the invention;

FIGS. 2A-2C are illustrations of a cannula and tensioning member for use in harvesting a vessel section in some embodiments of the invention;

FIGS. 3A-3C are illustrations of a drive system for use in harvesting vessel sections in some embodiments of the invention;

FIGS. 4A-4B are illustrations of a vessel tensioning handle for use in harvesting a vessel section in some embodiments of the invention;

FIGS. 5A-5B are further illustrations of the vessel tensioning handle of FIGS. 4A-4B;

FIG. 6 is an illustration of a bobbin spring assembly for use in the vessel tensioning handle of FIGS. 4A-5B;

FIGS. 7A-7B are illustrations of a self force regulating vacuum piston for use in harvesting a vessel section in some embodiments of the invention;

FIG. 8 is an illustration of a snap-back mechanism for use with the bobbin spring assembly of FIG. 6; and

FIGS. 9A-9B are flow diagrams of vessel harvesting methods in accordance with some embodiments of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

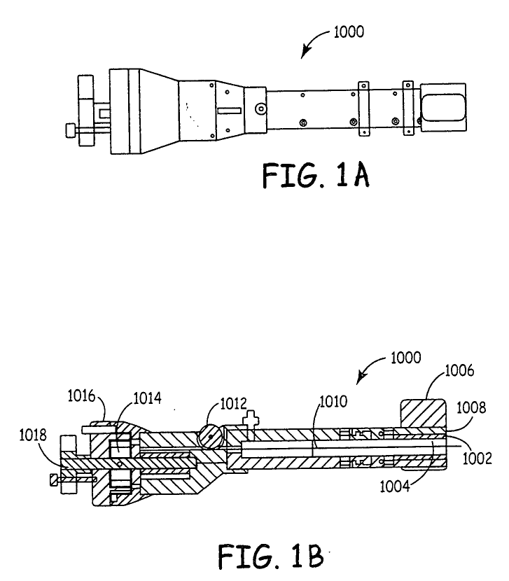

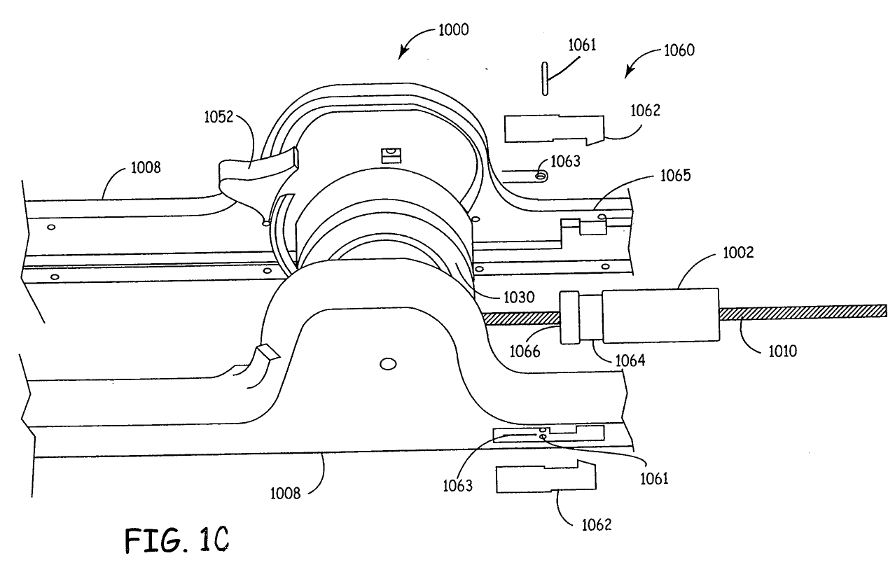

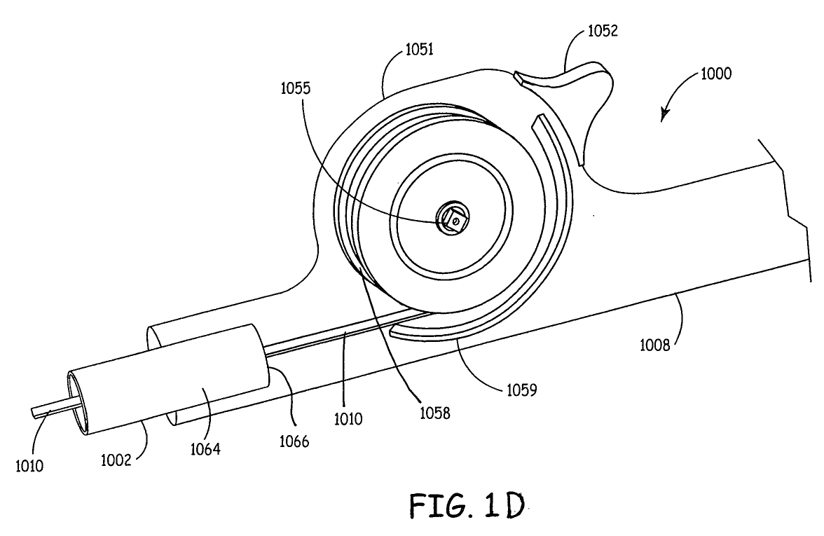

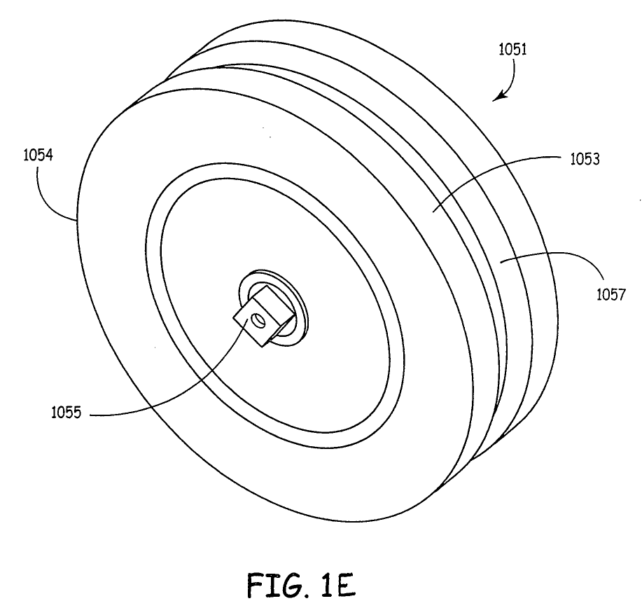

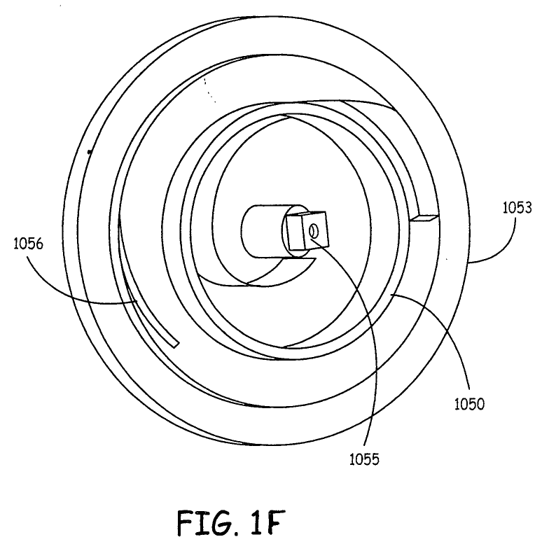

FIGS. 1A-1B illustrate a vessel tensioning handle 1000 for use in harvesting a vessel section according to some embodiments of the invention. The handle 1000 can include of a cutting tubular member 1002, an inner vessel stabilizer tube 1004, an outer knife blade turning collar 1006, a handle body 1008, a vessel tensioning cable or tape 1010, a tensioning cable clamping wheel 1012, a tensioning spring 1014, a spring tensioning adjuster 1016, and a tensioning control knob 1018.

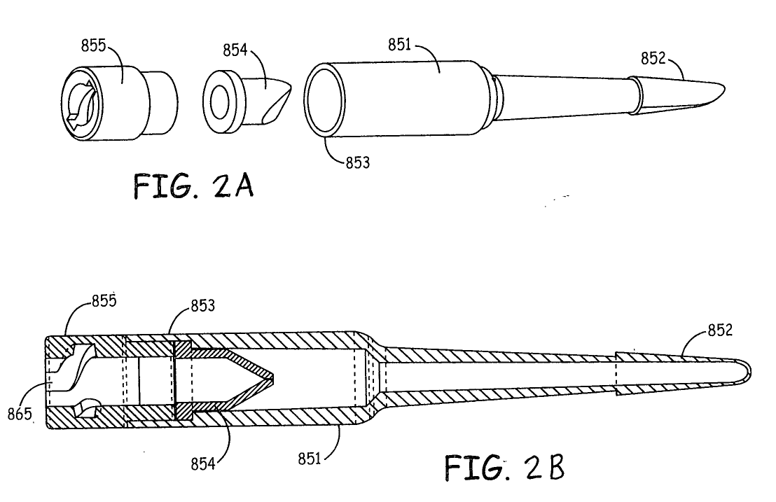

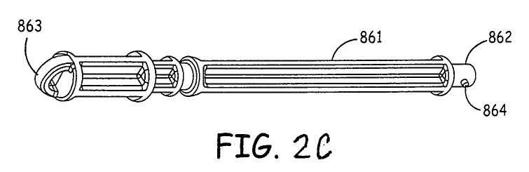

In one embodiment, the distal end of the vessel tensioning cable 1010 can be coupled to a proximal end of a tensioning device member 861, as shown in FIG. 2C. In one embodiment, the proximal end 853 of a cannula 851 can include a tension-coupling member 855, as shown in FIGS. 2A-2B, for coupling the vessel tensioning cable 1010 to the cannula 851. In one embodiment, a twist lock mechanism can be used to secure the tensioning device member 861 to the cannula 851. The distal end 862 of tensioning device member 861 is inserted, twisted and locked into place within the tension-coupling member 855 located at the proximal end 853 of the cannula 851. In one embodiment, a bayonet fastener mechanism can be used to couple the tensioning device member 861 to the tension coupling member 855. For example, raised bumps 864 sized to fit within grooves 865 can be used to couple the tensioning device member 861 to the tension-coupling member 855. The vessel tensioning cable 1010 can be coupled to the tensioning device member 861 at its proximal end 863, as shown in FIG. 2C.

FIGS. 1C-1F illustrate that the handle 1000 can include a spring bobbin assembly 1051, a brake 1052, and a locking assembly 1060. The handle 1000 can be used to couple the proximal end of the cutting tubular member 1002 to the handle body 1008, which includes the vessel tensioning cable 1010, the spring bobbin assembly 1051, the brake 1052, and the locking assembly 1060. In one embodiment, the distal end of the vessel tensioning cable 1010 can be coupled to the proximal end 863 of the tensioning device member 861. The tensioning device member 861 can supply a predetermined amount of pressure, e.g., 12 ounces, on the cannula 851, and thus, the vessel. This pressure on the cannula 851 and the vessel can be used to hold the vessel section to be harvested in its original starting position as the cutting tubular member 1002 is advanced over the vessel.

In one embodiment, as shown in FIG. 1E, the bobbin assembly 1051 includes a bobbin spring 1050, bobbin members 1053 and 1054, and a bobbin shaft 1055. As shown in FIG. 1D, the bobbin shaft 1055 couples the bobbin assembly 1051 to the handle body 1008. The proximal end of the vessel tensioning cable 1010 can be fixedly attached to the bobbin assembly 1051 by being placed within a slot 1056, as shown in FIG. 1F. The vessel tensioning cable 1010 can be wound around the bobbin assembly 1051 within a recess 1057, as shown in FIG. 1E. As shown in FIG. 1F, the bobbin spring 1050 can supply a tensioning force to the vessel tensioning cable 1010. As shown in FIG. 1D, the distal end 1059 of the tensioning brake 1052 can apply a variable force against the vessel tensioning cable 1010. The force applied by the brake 1052 can push the vessel tensioning cable 1010 against and into notches or grooves 1058, as shown in FIG. 1D. Pressing down on a proximal portion of the brake 1052 can translate into a greater force being applied to the vessel tensioning cable 1010 via the distal portion of the brake 1052.

In one embodiment, as shown in FIG. 1C, the locking assembly 1060 includes two pins 1061, two locking members 1062, and two springs 1063. The locking members 1062 and the springs 1063 are coupled to the handle body 1008 via the pins 1061. The springs 1063 bias the locking members 1062 into a locking position with the proximal end of the cutting tubular member 1002. Portions of the locking members 1063 fit into a recess 1064 located at the proximal end of the cutting tubular member 1002. The proximal end of the cutting tubular member 1002 also includes slots 1066. Raised portions 1065 of the handle body 1008 fit into the slots 1066, thereby preventing the rotation of the cutting tubular member 1002 with respect to the handle body 1008. In other words, rotation of the handle body 1008 translates to a rotation of the cutting tubular member 1002.

The vessel can be kept taught in order to not allow the vessel to start to migrate forward with the handle 1000 as this subjects the vessel to bunch, which can lead to cutting the main portion of the vessel. The vessel tensioning cable 1010 is used to hold the vessel in position while the cutting tubular member 1002 is advanced over the vessel. In some embodiments, this tensioning function can improve the quality of the harvested vessel.

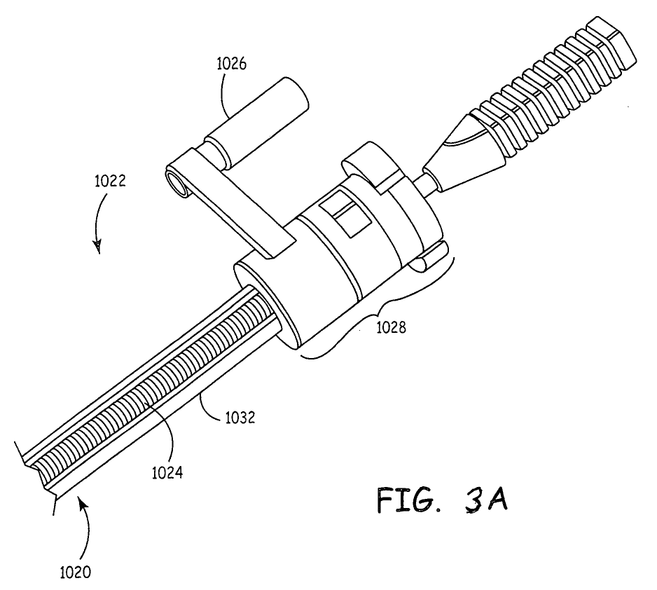

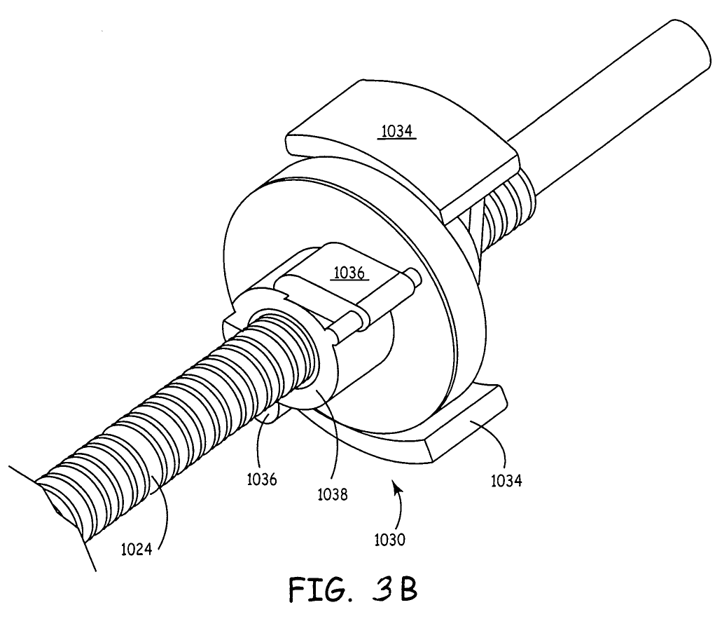

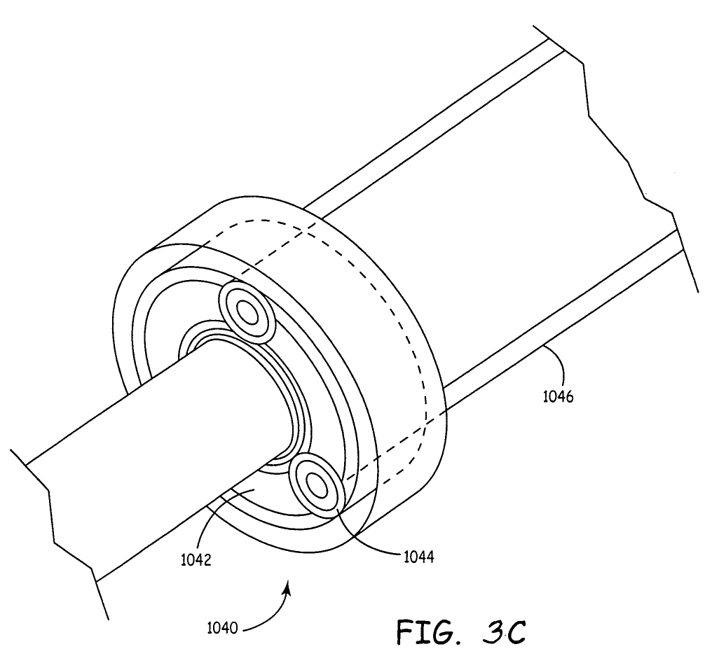

FIGS. 3A-3C illustrate a drive and release system 1030 according to embodiments of the invention. In some embodiments, the drive and release system 1030 can amplify user input (e.g., rotation) to a main body 1020 of a tensioning device 1022. In some embodiments, the drive system can assist advancement of the tensioning device 1022 given user input (e.g., rotation). The tensioning device 1022 for amplifying user input can include a planetary gear arrangement that provides an angular increase of approximately double the input. Altering gear systems 1028 can attain other input/output ratios. The tensioning device 1022 to provide device advancement utilizes a ratcheting mechanism on a lead screw 1024 to move a cutting tubular member 1032 forward. With the ratcheting feature, the tensioning device 1022 can be advanced during the clockwise rotation of a grip 1026. Counter-clockwise rotation can provide a secondary cutting stroke and can reset the ratchet mechanism for the next advancing stroke.

In the drive and release system 1030, push buttons 1034 can be pressed together by the operator to release a nut 1038 from a thread 1024 for free movement of the cutting tubular member 1032 along the thread 1024. In one embodiment, a vessel stabilization channel can traverse through the thread 1024. Clockwise rotation can advance the drive and release system 1030 along the thread 1024. The pitch of the thread 1024 and the angle of rotation can determine the travel distance. Counterclockwise rotation can reset one or more pawls 1036 on the nut 1038 to prepare for the next advance. Counterclockwise rotation can also provide a second swipe of the cutting tubular member 1032 across the tissue before the next advance. The pawls 1036 can be spring loaded against the nut 1038. The pawls 1036 can be disengaged for manual operation. FIG. 3C illustrates a planetary gear system 1040 having a pinion gear 1042 and ring gears 1044. The planetary gear 1040 can be located at the end of a handle 1046.

Some embodiments of the invention include a vessel tensioning handle that applies a substantially constant tension to a vessel being harvested from the surrounding tissue during a harvesting procedure. Some embodiments of the vessel tensioning handle can be operated by a clinician with a single hand. The tension that is applied to the vessel during the harvesting procedure can be of sufficient force to hold the vessel close to its original (i.e., native, pre-harvest) position, while at the same time minimizing damage to the vein during the harvest procedure.

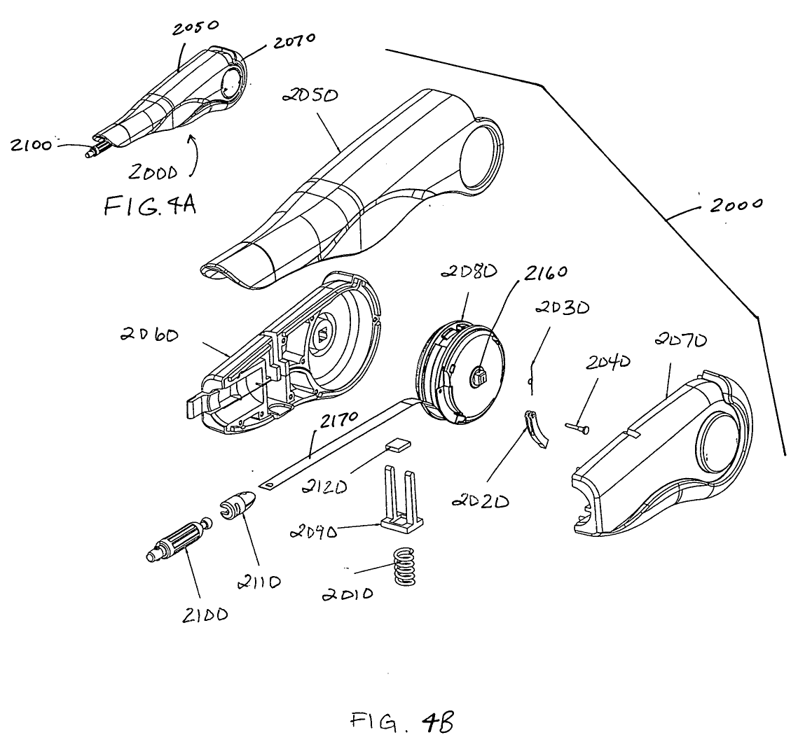

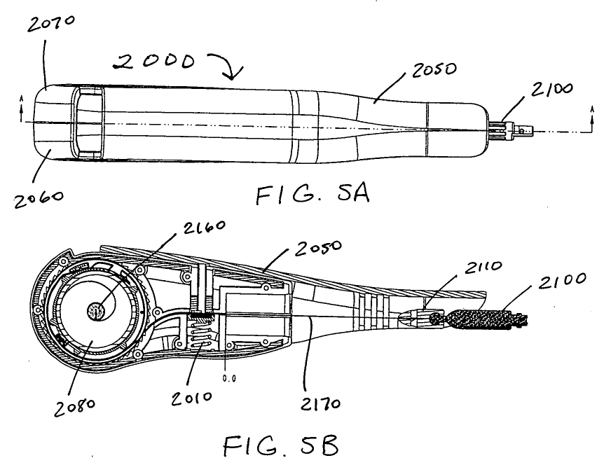

FIGS. 4A-5B illustrate one embodiment of a vessel tensioning handle 2000. The handle 2000 can include a brake spring 2010, a latch 2020, a spring 2030, a pin 2040, a brake handle 2050, a first housing 2060, a second housing 2070, a bobbin spring assembly 2080, a brake 2090, a ball cannula adaptor (or tensioning device member) 2100, a socket cannula adapter 2110, a brake pad 2120, and a vessel tensioning tape 2170. The first housing 2060 can be coupled to the second housing 2070 in order to enclose the bobbin spring assembly 2080. A suitable bearing can be used to allow the bobbin spring assembly 2080 to rotate within the first housing 2060 and the second housing 2070. The first housing 2060 and the second housing 2070 can include various bosses and recesses suitable to guide the vessel tensioning tape 2170 back to the bobbin spring assembly 2080 during retraction. The latch 2020, the spring 2030, and the pin 2040 can be used to help reduce rapid retraction of the vessel tensioning tape 2170, as further described with respect to FIG. 8.

The brake handle 2050 can be coupled to the first housing 2060 and the second housing 2070 by the brake handle 2050 receiving circular bosses of the first housing 2060 and the second housing 2070 within corresponding circular recesses on each lateral side of the brake handle 2050. The brake handle 2050 can pivot about the circular bosses in order to move the brake 2090 until the brake 2090 engages and holds the vessel tensioning tape 2170 between the brake pad 2120 and the brake 2090. The brake spring 2010 biases the brake pad 2120 toward the brake 2090 and the brake handle 2050.

A distal end of the vessel tensioning tape 2170 can be coupled to the socket cannula adapter 2110. The socket cannula adapter 2110 can include a ball socket that receives a ball of the ball cannula adaptor (or tensioning device member) 2100, which can allow the ball cannula adapter 2100 and the cannula 851 (as shown in FIGS. 2A-2B) to rotate with respect to the vessel tensioning tape 2170 as the vessel is pulled through the tubular cutting device.

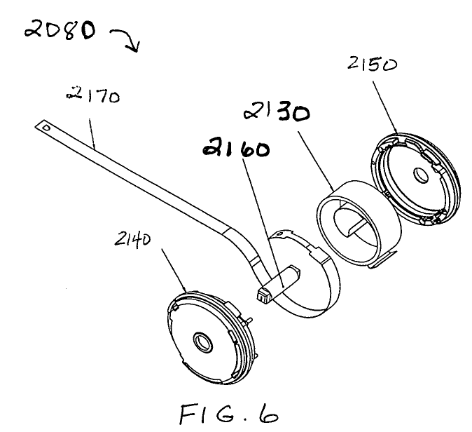

FIG. 6 further illustrates the bobbin spring assembly 2080. In one embodiment, the bobbin spring assembly 2080 includes a coil tensioner 2130, a first housing side 2140, a second housing side 2150, a bobbin shaft 2160, and the vessel tensioning tape 2170. The bobbin shaft 2160 can be secured within recesses in the first housing 2060 and the second housing 2070. In one embodiment, the bobbin shaft 2160 can have a square cross-section. The first housing side 2140 and the second housing side 2150 also be coupled to the bobbin shaft 2160 and can enclose the coil tensioner 2130. The coil tensioner 2130 can exert tension on the vessel tensioning tape 2170 in order to pull and wrap the vessel tensioning tape 2170 around the bobbin spring assembly 2080. A proximal end of the vessel tensioning tape 2170 can include recesses and/or apertures that can be used to secure the vessel tensioning tape 2170 to the bobbin spring assembly 2080.

Some embodiments of the invention provide a vessel tensioning handle including a snap-back mechanism designed to substantially reduce or eliminate the rapid and/or uncontrolled retraction of the vessel tensioning tape back into the handle. Such release can occur, for example, when the pulling tension is released, e.g., when the vessel tensioning tape detaches from the vessel being harvested, or when the brake in the handle releases while the vessel tensioning tape is unattached. In some embodiments, the snap-back mechanism includes a weighted arm and a balance spring that can act to protect a harvested vessel in the event of an uncontrolled retraction of the vessel tensioning tape back into the handle.

FIG. 8 illustrates a snap-back mechanism 2600 according to one embodiment of the invention. The snap-back mechanism 2600 can be a component of the bobbin spring assembly 2080 (as shown in FIGS. 4A-5B) and can be positioned within the handle 2000 (as shown in FIG. 5B). In one embodiment, the snap-back mechanism 2600 is activated by centripetal force. In some embodiments, as shown in FIG. 8, the snap-back mechanism 2600 includes a housing 2610, a bobbin assembly 2620, teeth 2630, an arm 2640, a spring 2650, and an arm-retaining pin 2660. The spring 2650 can be, for example, a light spring. The spring 2650 can control the speed at which the arm 2640 is forced away from the center of the bobbin assembly 2620. In one embodiment, the teeth 2630 are molded into the side of the housing 2610. When the arm 2640, the spring 2650, and the arm-retaining pin 2660 are assembled into the bobbin assembly 2620, the arm 2640 is free to move on the arm-retaining pin 2660 and is held toward the center of the bobbin assembly 2620 by the spring 2650. The spring 2650 is strong enough to hold the arm 2640 in position until the rotational speed of the bobbin assembly 2620 reaches an upper limit, at which time the centripetal force of the bobbin assembly 2620 causes the arm 2640 to move outward from the center of the bobbin assembly 2620. When the arm 2640 moves away from the center of the bobbin assembly 2620, the pointed end of the arm 2640 contacts the teeth 2630 and the bobbin assembly 2620 stops turning. The end of the arm 2640 and the teeth 2630 in the tensioner housing 2610 are designed to lock together. The bobbin assembly 2620 can be released by pulling on the vessel tensioning tape 2170, which causes the arm 2640 to be released from the teeth 2630 by the spring 2650 pulling the arm 2640 back toward the center of the bobbin assembly 2620.

As discussed above, the spring 2650 holds the arm 2640 in position until the rotational speed of the bobbin assembly 2620 reaches an upper limit, at which time the arm 2640 locks with the teeth 2630. However, if the upper limit it not reached, the snap-back mechanism 2600 will not be activated, and the entire length of the vessel tensioning tape 2170 can be used during the vessel harvesting procedure.

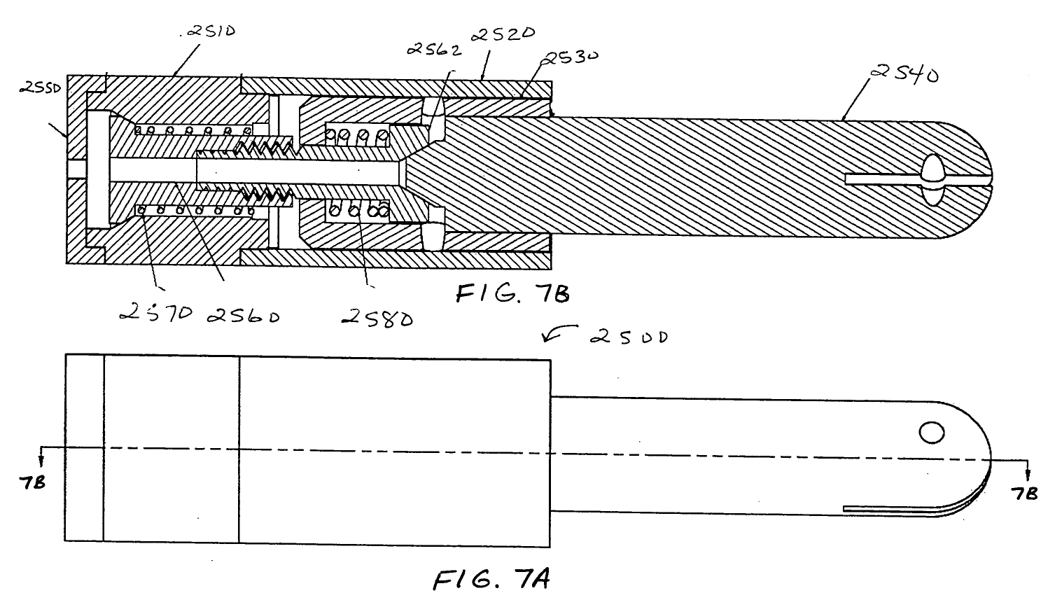

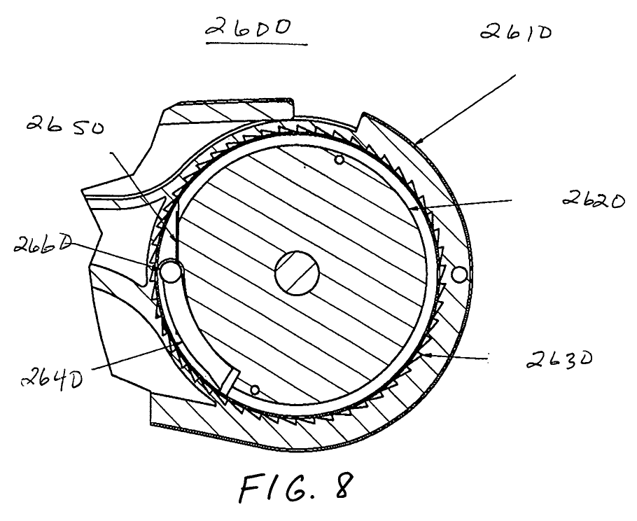

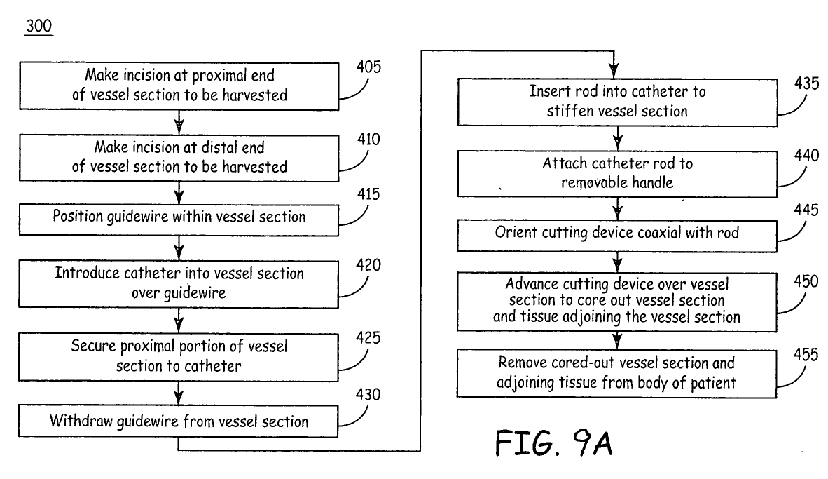

FIGS. 7A and 7B illustrate a self-force regulating vacuum piston system 2500 that can be used to apply tension to a vessel when harvesting vessel sections in some embodiments of the invention. The vacuum piston system 2500 can include a primary piston body 2510, a primary body extension 2520, and a ported piston cap 2550. The vacuum piston system 2500 can include two internal vacuum control valves—a primary vacuum seal valve 2560 and a secondary seal valve 2562 that are controlled by a pull force exerted on a cannula mount 2540. For example, when a vacuum is applied to one side of the primary piston body 2510, a pulling force is transferred to the end of the primary piston body 2510 coupled to the cannula mount 2540. The pulling force can be started and regulated by the valves 2560, 2562 (along with primary vacuum seal spring 2570). A vacuum can be applied to the top side of the primary piston body 2510, and can be bled through the primary piston body 2510 by the primary vacuum seal valve 2560. When enough manual pull is applied to the cannula mount 2540, the primary vacuum seal valve 2570 can be pulled closed, and the vacuum can build up on top of the primary piston body 2510. A force is then applied to the end of the primary piston body 2510 coupled to the cannula mount 2540. In one embodiment, the secondary seal valve 2562 can be set to open (in order to release the vacuum) when the pull on the primary piston body 2510 exceeds about 12 to 14 ounces. When more than about 12 to 14 ounces of pull is applied to the end of the primary piston body 2510 coupled to the cannula mount 2540, the vacuum is released by the secondary seal valve 2530 (with vacuum release seal spring 2580), which reduces the amount of pull that can be generated by the primary piston body 2510.

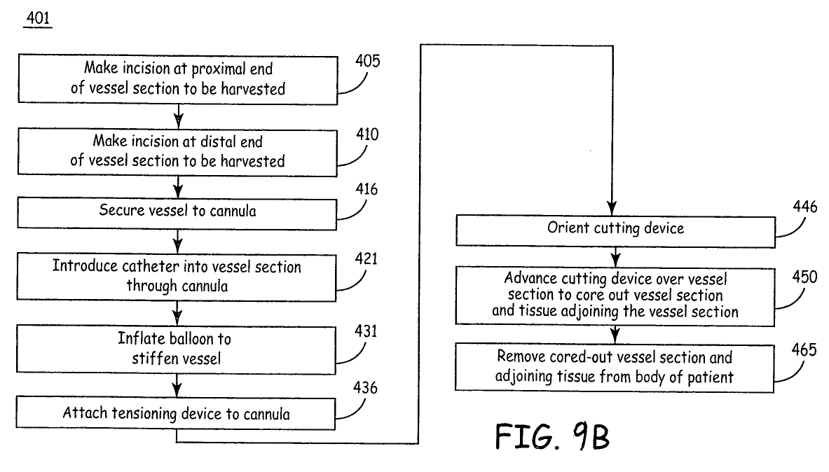

FIG. 9A is a flow diagram of a vessel harvesting method according to one embodiment of the invention. In this embodiment, a first incision is made at a point corresponding to a proximal end of the vessel section to be harvested (Block 405). A second incision is made at a point corresponding to a distal end of the vessel section (Block 410). A guidewire is then positioned within the vessel section (Block 415). Alternatively, the guidewire can be inserted into the vessel before the second incision is made. Inserting the guidewire prior to making the second incision can aid in determining the optimal location for the second incision. Once the second incision has been made, the guidewire is positioned such that it extends beyond and outside of the vessel section at both the distal and proximal ends of the section.

A catheter is introduced into the vessel section over the previously placed guidewire (Block 420). A proximal portion of the vessel section is secured to the catheter (Block 425), for example by suturing the vessel onto a barb positioned adjacent to the proximal end of the catheter. Alternatively, the catheter can be introduced into the vessel without a guidewire being previously placed.

The guidewire (if present) is withdrawn (Block 430), and a rod can be inserted into the catheter to stiffen the vessel section (Block 435). Both the catheter and the rod can be attached to a removable handle (Block 440). The handle can carry a tubular cutting device, or the cutting device can be introduced over the handle after the handle has been attached to the catheter and rod. An inner lumen of the cutting device provides a close-sliding fit for the handle. The tubular cutting device is thus oriented coaxial with the rod and with the vessel section to be harvested (Block 445).

The cutting device is then advanced over the vessel section to core out the vessel section and tissue adjoining the vessel section (Block 450). The cutting device can be advanced by either pushing or pulling the device over the vessel section. Where the cutting device comprises two tubular members, one positioned within the other, the two tubular members can be advanced separately. For example, inner tubular member 120 can be advanced first to hold the vessel and surrounding tissue, while outer tubular member 110 is advanced second to cut the tissue being held by the inner tubular member. The process of incrementally advancing the inner tubular member and then the outer tubular member is repeated until the entire section has been excised. Advancing the inner tubular member ahead of the outer tubular member can protect the walls of the vessel from the cutting element(s) positioned on the outer tubular member. Advancing and rotating the inner and outer tubular members separately can also protect the side branches of the vessel by holding them in place to achieve a clean cut at a sufficient length. The cutting device, for example, can be twisted first in one direction and then in the other direction, or it can be rotated over the vessel. The outer and inner tubular members can be twisted in opposite directions to provide a scissoring action.

The cored out vessel section and adjoining tissue are removed from the body of the patient (Block 455). Either before or after removing the vessel section and adjoining tissue, a hemostatic control method for branch vessels severed as a result of coring out the vessel section can be introduced through either the first or the second incision. The hemostatic control method can be, for example, a biological sealant, e.g., platelet gel that can be prepared from the patient's blood and injected or otherwise introduced along the track of the cutting device. The hemostatic control method can also be a thrombogenic substance such as fibrinogen, fibrin and/or thrombin placed in the track left by the cutting device. Alternatively, or in combination with a biological sealant, a biocompatible or biodegradable tube can be enclosed within the cutting device to be delivered as the cutting device is advanced over the vessel or after the cutting device has completed coring out the vessel and adjoining tissue. The tube exerts pressure on the cut branch vessels and can be either removed or, in the case of a biodegradable tube, left to dissolve or degrade over a period of a few days, for example. The space left after the removal of the vessel can also be filled with gauze to provide internal pressure to limit bleeding and absorb blood. The gauze can be removed periodically to check for absorbed blood. Limited blood collected on the gauze indicates the wound bleeding has diminished.

Hemostatic control methods are not required for embodiments of the invention as the tubular cutting device itself can exert pressure on the cut branch vessels while it remains within the patient's body. A drain can be inserted at the end of the harvesting procedure to deal with any bleeding that does occur. The site of the vessel harvesting procedure, e.g., the leg of a patient, can also be wrapped with a compression bandage to limit bleeding.

In an alternative method of the invention, a rod is inserted directly into the vessel. Thus, no guidewire and/or catheter is used. In one embodiment, a proximal portion of the vessel can be attached to the rod rather than to the catheter as described above. The handle is then attached to the rod.

In another alternative method of the invention, the catheter is inserted directly into the vessel. Thus, no guidewire or rod is used. In one embodiment, the catheter includes one or more inflatable structures, such as balloons. In yet another alternative method of the invention, no catheter or rod is used; only a guide wire is used.

FIG. 9B is a flow diagram of a vessel harvesting method according to another embodiment of the invention. A first incision is made at a point corresponding to a proximal end of the vessel section to be harvested (Block 405). A second incision is made at a point corresponding to a distal end of the vessel section (Block 410). A cannula is then inserted into the proximal end of the vessel section, which is located near the knee. The proximal end of the vessel is then secured to the cannula (Block 416), for example by suturing the vessel onto a barb or raised portion positioned adjacent to the distal end of the cannula. A balloon catheter is then introduced through the cannula and positioned within the vessel section (Block 421). Once positioned, the balloon is inflated to stiffen the vessel section (Block 431). A vessel-tensioning device or system is then attached to the cannula to provide a vessel-tensioning force to the vessel section (Block 436).

A cutting device is oriented coaxially with the cannula, the balloon, and the vessel section to be harvested (Block 446). The cutting device is then advanced over the vessel section to core out the vessel section and tissue adjoining the vessel section (Block 450). The cutting device, for example, can be twisted first in one direction and then in the other direction, or it can be rotated over the vessel. The cored out vessel section and adjoining tissue are removed from the body of the patient (Block 455). Either before or after removing the vessel section and adjoining tissue, a hemostatic control method for treating branch vessels severed as a result of coring out the vessel section can be introduced through either the first or the second incision. The hemostatic control method can be, for example, a biological sealant, e.g., platelet gel that can be prepared from the patient's blood and injected or otherwise introduced along the track of the cutting device. The hemostatic control method can also be a thrombogenic substance such as fibrinogen, fibrin and/or thrombin placed in the track left by the cutting device. Alternatively, or in combination with a biological sealant, a biocompatible or biodegradable tube can be enclosed within the cutting device to be delivered as the cutting device is advanced over the vessel or after the cutting device has completed coring out the vessel and adjoining tissue. The tube exerts pressure on the cut branch vessels and can be either removed or, in the case of a biodegradable tube, left to dissolve or degrade over a period of a few days, for example. The space left after the removal of the vessel can also be filled with gauze to provide internal pressure to limit bleeding and absorb blood. The gauze can be removed periodically to check for absorbed blood. Limited blood collected on the gauze indicates the wound bleeding has diminished.

The site of the vessel harvesting procedure, e.g., the leg of a patient can be wrapped with a compression bandage to limit bleeding following vessel harvest. For example, a compression wrap specifically designed to apply direct pressure over a wound created by a device of the invention might be used. Such a compression wrap has a compartment, such as a pocket, to house a tubular object such as a tube, rolled gauze, rod, hemispherical tube, or the like. When the wrap is secured onto a patient's leg, this tubular/semi-tubular object is positioned directly over the wound, and bleeding is controlled. Thus, hemostasis is achieved. In one embodiment, the wrap is made out of an elastic material, such as neoprene, and can include hook and loop closures or similar such enclosure system so that an appropriate pressure is applied to the patient's leg once it is enclosed by the wrap. When open the wrap lies flat, so that it can be placed under the patient's leg prior to the vessel harvesting. Immediately following vessel harvest, the compression wrap with tubular object is positioned onto the patient's leg, making sure that the tubular object is placed directly over the wound so that direct pressure to the wound can be applied.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

1. A vessel tensioning handle for use with a cutting device and a cannula for harvesting a section of a vessel, the cannula being coupled to the vessel, the handle comprising:
a housing adapted to be coupled to the cutting device;
a bobbin assembly positioned within the housing;
a vessel tensioning tape coupled to the bobbin assembly; and
a tensioning device member coupled to the vessel tensioning tape, the tensioning device member adapted to be coupled to the cannula, the vessel tensioning tape placing tension on the section of the vessel as the cutting device is advanced over the vessel.

2. The handle of claim 1 and further comprising a brake and wherein the housing includes a moveable brake handle adapted to engage the brake.

3. The handle of claim 1 wherein the bobbin assembly includes a first housing side, a second housing side, a bobbin shaft, and a coil tensioner.

4. The handle of claim 1 wherein the bobbin assembly includes a snap-back mechanism to prevent rapid retraction of the vessel tensioning tape.

5. The handle of claim 4 wherein the snap-back mechanism includes teeth molded in a portion of a housing, an arm that engages the teeth, a spring that biases the arm, and an arm-retaining pin about which the arm pivots.

6. A method of harvesting a section of a vessel from surrounding tissue, the method comprising:
making a first incision at a proximal end of the section of the vessel;
making a second incision at a distal end of the section of the vessel;
inserting a cannula into the proximal end of the vessel;
securing the proximal end of the vessel to the cannula;
coupling a vessel tensioning tape to the cannula;
applying tension to the vessel; and
advancing a cutting device over the vessel.

7. The method of claim 6 and further comprising retracting the vessel tensioning tape into a handle.

8. The method of claim 7 and further comprising coring out the vessel from the surrounding tissue as the vessel tensioning tape is retracted into the handle.

9. The method of claim 7 and further comprising applying a brake to stop retracting of the vessel tensioning tape.

10. The method of claim 7 and further comprising applying tension to the vessel tensioning tape to cause the vessel tensioning tape to retract into the handle.

11. The method of claim 6 and further comprising coupling the vessel tensioning tape to the cannula with a twisting motion.

12. The method of claim 6 and further comprising guiding the cutting device with a balloon catheter while retracting the vessel tensioning tape.

13. The method of claim 6 and further comprising pulling the cannula and the vessel through the cutting device while retracting the vessel tensioning tape.

14. The method of claim 6 and further comprising amplifying a cutting force applied to the handle.

15. The method of claim 6 and further comprising assisting advancement of the cutting device along the vessel.

16. The method of claim 6 and further comprising applying a substantially constant tension to the vessel as the vessel is harvested.

17. The method of claim 6 and further comprising applying tension to the vessel by retracting the vessel tensioning tape around a bobbin assembly in a handle.

18. The method of claim 6 and further comprising applying tension to the vessel with a piston assembly.

19. The method of claim 6 and further comprising reducing rapid retraction of the vessel tensioning tape into a handle.

20. The method of claim 18 and further comprising reducing rapid retraction of the vessel tensioning tape by centripetal force causing an arm to engage teeth in a housing.

21. The method of claim 6 and further comprising manipulating a handle coupled to the vessel tensioning tape in order to manipulate the cutting device.
